(19) United States
(12) Patent Application Publication
(10) Pub. No.: US 2025/0010274 A1
(43) Pub. Date: Jan. 9, 2025

(54) CATALYST FOR METHACRYLIC ACID PRODUCTION, METHOD FOR PRODUCING SAME, AND METHOD FOR PRODUCING METHACRYLIC ACID AND METHACRYLIC ACID ESTERS USING CATALYST

(71) Applicant: MITSUBISHI CHEMICAL CORPORATION, Tokyo (JP)

(72) Inventors: Shogo HAYAKAWA, Tokyo (JP); Jun HIRATA, Tokyo (JP); Mitsuru KANNO, Tokyo (JP); Wataru NINOMIYA, Tokyo (JP)

(73) Assignee: MITSUBISHI CHEMICAL CORPORATION, Tokyo (JP)

(21) Appl. No.: 18/891,889

(22) Filed: Sep. 20, 2024

Related U.S. Application Data
(63) Continuation of application No. PCT/JP2023/011498, filed on Mar. 23, 2023.

(30) Foreign Application Priority Data
Mar. 24, 2022 (JP) 2022-048075

(51) Int. Cl. B01J 23/28; B01J 23/22; B01J 23/30; B01J 23/72; B01J 37/04; B01J 37/08; C07C 51/16

(52) U.S. Cl. CPC B01J 23/28; B01J 23/22; B01J 23/30; B01J 23/72; B01J 37/04; B01J 37/08; C07C 51/16; C07C 2523/22; C07C 2523/28; C07C 2523/30; C07C 2523/72

(57) ABSTRACT

An object is to provide a catalyst which has high heat resistance and which can allow for production of methacrylic acid at a high yield and to provide methods for producing methacrylic acid and methacrylic acid ester with the catalyst, and the object is achieved by use of a catalyst having a specified peak in powder X-ray measurement.

### CROSS-REFERENCE TO RELATED APPLICATION

This is a continuation of International Application PCT/JP2023/011498, filed on Mar. 23, 2023, and designated the U.S., and claims priority from Japanese Patent Application 2022-048075 which was filed on Mar. 24, 2022, the entire contents of which are incorporated herein by reference.

### TECHNICAL FIELD

The present invention relates to a catalyst for methacrylic acid production, a method for producing the catalyst, and methods for producing methacrylic acid and methacrylic acid ester with the catalyst.

### BACKGROUND ART

There are known as catalysts for methacrylic acid production, for use in the production of methacrylic acid by oxidation of methacrolein (hereinafter, also simply designated as “catalysts”), for example, heteropoly acid-based catalysts each including molybdenum and phosphorus. Heteropoly acid refers to condensed oxyacid with 12 coordinating atoms forming a basic structure of poly acid (hereinafter, designated as “polyatoms”) and oxide of heteroatom. Heteropoly acid-based catalysts include proton-type heteropoly acid with protons as counter cations, and heteropoly acid salts with some protons substituted with cations other than such protons (hereinafter, proton-type heteropoly acid is also simply expressed as “heteropoly acid”, and at least one selected from proton-type heteropoly acid and heteropoly acid salts is also simply expressed as “heteropoly acid (salt)”).

NPL 1 describes with respect to structures of heteropoly acid (salt), phosphorus, silicon, arsenic, germanium, titanium, antimony, and the like which can serve as heteroatoms, and tungsten, molybdenum, vanadium, niobium, tantalum, and the like which can serve as polyatoms of heteropoly acid (salt). Keggin-type, Dawson-type, Preyssler-type, and the like are also described as basic structures of heteropoly acid (salt).

PTL 1 discloses, as a catalyst for methacrylic acid production, a catalyst including a Keggin-type heteropoly acid salt having a composition represented by the following formula:

PₐMo_{b}V_{c}X_{d}YₑO_{f}

wherein P, Mo, V and O respectively represent phosphorus, molybdenum, vanadium and oxygen, X represents at least one element selected from potassium, rubidium, cesium and thallium, Y represents at least one element selected from copper, arsenic, antimony, boron, silver, bismuth, iron, cobalt, lanthanum and cerium, a, b, c, d, e and f respectively represent the atomic ratios of P, Mo, V, X, Y and O, and when b=12 is set, a, c, d and e are each independently a value of more than 0 and 3 or less, and f is a value defined by the oxidation state and the atomic ratio of each element other than oxygen.

Examples of known Keggin-type heteropoly acid include H₃PMo₁₂O₄₀, H₄PMo₁₁VO₄₀H₃, and PW₁₂O₄₀, and NPL 1 indicates that the pyrolysis temperature of PW₁₂O₄₀ is higher than those of H₃PMo₁₂O₄₀ and H₄PMo₁₁VO₄₀H₃. In this regard, as described in NPL 2, use of H₃PMo₁₂O₄₀ as a catalyst for methacrylic acid production favorably provides methacrylic acid, but use of PW₁₂O₄₀ leads to both activity and selectivity which are very low.

### RELATED ART DOCUMENTS

### Patent Documents

PTL 1: JP-A 2003-010691

### Non Patent Documents

NPL 1: Toshio Okuhara, Noritaka Mizuno, Makoto Misono, Advances in Catalysis, volume 41.

NPL 2: Shuhei Yasuda, Atsuki Iwakura, Jun Hirata, Mitsuru Kanno, Wataru Ninomiya, Ryoichi Otomo, Yuichi Kamiya, Catalysis Communications, 125 (2019), 43-47.

### SUMMARY OF THE INVENTION

However, known catalysts for methacrylic acid production are low in heat resistance and still insufficient in catalyst life. Therefore, it is demanded for use as an industrial catalyst to develop a catalyst which has high heat resistance and which can allow for production of methacrylic acid at a high yield.

An object of the present invention is to provide a catalyst which has high heat resistance and which can allow for production of methacrylic acid at a high yield. Another object of the present invention is to provide methods for producing methacrylic acid and methacrylic acid ester with the catalyst.

The present inventors have made intensive studies in view of the above problems, and as a result, have found that the above problems can be solved by use of a catalyst having a specified peak in powder X-ray measurement, and thus have completed the present invention.

Specifically, the present invention includes the following.

[1]: A catalyst for use in the production of methacrylic acid by oxidation of methacrolein, wherein
the catalyst contains heteropoly acid including phosphorus, molybdenum, and tungsten,
(a1) IB/IA is 0.015 or less when the intensity of a diffraction peak (peak A) at 2θ=26.1±0.2° is defined as IA and the intensity of a diffraction peak (peak B) at 2θ=23.0±0.2° is defined as IB in powder X-ray diffraction measurement at 25° C., and
(b) the lowest temperature T1 at which a diffraction peak (peak E) at 2θ=27.1±0.2° appears in powder X-ray diffraction measurement performed at an interval of 10° C. from 400° C. with a temperature rise at 10° C./min is 440° C. or more.

[2]: The catalyst for methacrylic acid production according to [1], wherein the IB/IA is 0.01 or less.

[3]: The catalyst for methacrylic acid production according to [1] or [2], wherein (a2) IC/IA is 0.0055 or less when the intensity of a diffraction peak (peak C) at 2θ=34.1±0.2° is defined as IC in powder X-ray diffraction measurement at 25° C.

[4]: The catalyst for methacrylic acid production according to [3], wherein the IC/IA is 0.0005 to 0.0055.

[5]: The catalyst for methacrylic acid production according to [3] or [4], wherein the IC/IA is 0.001 to 0.0053.

[6]: The catalyst for methacrylic acid production according to any of [1] to [5], wherein (a3) ID/IA is 0.003 or less when the intensity of a diffraction peak (peak D) at 2θ=48.3±0.2° is defined as ID in powder X-ray diffraction measurement at 25° C.

[7]: The catalyst for methacrylic acid production according to any of [1] to [6], wherein the T1 is 440 to 500° C.

[8]: The catalyst for methacrylic acid production according to any of [1] to [7], having a composition represented by the following formula (I):

PₐMo_{b}W_{c}V_{d}CuₑA_{f}E_{g}GₕOᵢ (I)

wherein P, Mo, W, V, Cu and O respectively represent phosphorus, molybdenum, tungsten, vanadium, copper and oxygen, A represents at least one element selected from the group consisting of antimony, bismuth, arsenicum, germanium, tellurium, selenium and silicon, E represents at least one element selected from the group consisting of iron, zinc, chromium, tantalum, cobalt, nickel, manganese, titanium and niobium, G represents at least one element selected from the group consisting of potassium, rubidium and cesium, a to i each represent the molar ratio of each component, b+c=12, a=0.5 to 3, c=0.22 to 5, d=0.01 to 3, e=0.01 to 2, f=0 to 3, g=0 to 3, and h=0.01 to 3 are satisfied, and i is the molar ratio of oxygen necessary for satisfying the valence of such each component.

[9]: The catalyst for methacrylic acid production according to [8], wherein c=0.22 to 3 is satisfied in the formula (I).

[10]: A method for producing the catalyst for methacrylic acid production according to any one of [1] to [9], the method including:
(i) a step of mixing a phosphorus raw material, a molybdenum raw material and a tungsten raw material with a solvent, to prepare a solution or slurry (liquid A) having a pH of 0.1 to 4;
(ii) a step of drying the liquid A, to obtain a dried product; and
(iii) a step of firing the dried product, to obtain a fired product.

[11]: The method for producing the catalyst for methacrylic acid production according to [10], wherein a tungsten raw material having a solubility at 20° C. of 4.1 g/100 mL or more occupies 50% by mass or more of the entire tungsten raw material in step (i).

[12]: The method for producing the catalyst for methacrylic acid production according to or [11], wherein the pH of the liquid A is 0.1 to 3 in step (i).

[13]: A method for producing methacrylic acid, including a step of oxidizing methacrolein with the catalyst for methacrylic acid production according to any of [1] to [9], to produce methacrylic acid.

[14]: A method for producing methacrylic acid, including a step of oxidizing methacrolein with a catalyst for methacrylic acid production, produced by the method according to any one of [10] to [12], to produce methacrylic acid.

[15]: A method for producing methacrylic acid ester, comprising a step of esterifying methacrylic acid produced by the method according to [13] or [14].

### EFFECTS OF THE INVENTION

According to the present invention, there can be provided a catalyst which has high heat resistance and which can allow for production of methacrylic acid at a high yield.

### MODE FOR CARRYING OUT THE INVENTION

Hereinafter, embodiments according to the present invention are described, but the present invention is not limited to the following. Herein, a numerical value range expressed with “to” means a range including numerical values described before and after “to” respectively as the lower limit value and the upper limit value, and “A to B” means A or more and B or less. While the invention has been described in detail and with reference to specific embodiments thereof, it will be apparent to one skilled in the art that various changes and modifications can be made therein without departing from the spirit and scope thereof.

### Catalyst for Methacrylic Acid Production

A catalyst for methacrylic acid production according to the present embodiment is a catalyst for use in the production of methacrylic acid by oxidation of methacrolein, wherein the catalyst contains heteropoly acid including phosphorus, molybdenum and tungsten, and the following conditions (a1) and (b) are satisfied.

(a1) IB/IA is 0.015 or less when the intensity of a diffraction peak (peak A) at 2θ=26.1±0.2° is defined as IA and the intensity of a diffraction peak (peak B) at 2θ=23.0±0.2° is defined as IB in powder X-ray diffraction measurement at 25° C.

(b) The lowest temperature T1 at which a diffraction peak (peak E) at 2θ=27.1±0.2° appears in powder X-ray diffraction measurement performed at an interval of 10° C. from 400° C. with a temperature rise at 10° C./min is 440° C. or more.

Such a catalyst has high heat resistance and can allow for production of methacrylic acid at a high yield.

The catalyst for methacrylic acid production according to the present embodiment preferably further satisfies the following condition (a2) or (a3), more preferably satisfies (a2) and (a3).

(a2) IC/IA is 0.0055 or less when the intensity of a diffraction peak (peak C) at 2θ=34.1±0.2° is defined as IC in powder X-ray diffraction measurement at 25° C.

(a3) ID/IA is 0.003 or less when the intensity of a diffraction peak (peak D) at 2θ=48.3±0.2° is defined as ID in powder X-ray diffraction measurement at 25° C.

### Powder X-Ray Diffraction Measurement of Catalyst

(Conditions (a1) to (a3))

When the catalyst containing heteropoly acid including phosphorus, molybdenum and tungsten is subjected to powder X-ray diffraction measurement at 25° C., a diffraction peak (peak A) observed at 2θ=26.1±0.2° and a diffraction peak (peak B) observed at 2θ=23.0±0.2° are each a peak mainly originating from a heteropoly acid structure. Specifically, the peak A mainly originates from a structure having phosphorus as heteroatoms and molybdenum as 10 or more of polyatoms (hereinafter, also designated as “structure A”). The peak B mainly originates from a structure having phosphorus as heteroatoms, any other elements than molybdenum, such as tungsten and vanadium, as 6 to 11 of polyatoms, and molybdenum as the balance (hereinafter, also designated as “structure B”). The structure B causes deterioration in activity in oxidation reaction of methacrolein.

A diffraction peak (peak C) observed at 2θ=34.1±0.2° and a diffraction peak (peak D) observed at 2θ=48.3±0.2° are each a peak mainly originating from an oxide structure constituted from elements forming no heteropoly acid structure. Specifically, the peak C mainly originates from a molybdenum-containing oxide structure. The peak D mainly originates from a tungsten-containing oxide structure. Such oxides promote sequential oxidation in oxidation reaction of methacrolein and cause deterioration in selectivity of methacrylic acid.

The IB/IA is 0.015 or less when the intensity of the peak A is defined as IA and the intensity of the peak B is defined as IB in powder X-ray diffraction measurement at 25° C. of the catalyst for methacrylic acid production according to the present embodiment. It is considered that the content of the structure B unsuitable for methacrylic acid production is here sufficiently low to provide a favorable yield of methacrylic acid. The upper limit of the IB/IA is preferably 0.01 or less, more preferably 0.005 or less.

The IC/IA is preferably 0.0055 or less when the intensity of the peak C is defined as IC. It is considered that the content of molybdenum-containing oxide unsuitable for methacrylic acid production is here sufficiently low to provide a favorable yield of methacrylic acid. The upper limit of the IC/IA is more preferably 0.0053 or less, further preferably 0.0052 or less. The lower limit of the IC/IA is preferably 0.0005 or more, more preferably 0.001 or more from the viewpoint of an enhancement in activity of methacrolein oxidation reaction.

Examples of the method for providing a catalyst having IB/IA and IC/IA respectively falling within prescribed ranges include a method including production of the catalyst by a method including steps (i) to (iii) described below and adjustment of the amount of the tungsten raw material used in step (i) and the pH of the resulting liquid A.

When the intensity of the peak D is defined as ID, the ID/IA is preferably 0.003 or less, more preferably 0.002 or less, further preferably 0.001 or less.

Examples of the method for providing a catalyst having ID/IA within a prescribed range include a method including production of the catalyst by a method including steps (i) to (iii) described below and use of 50% by mass or more of a tungsten raw material having a solubility at 20° C. of 4.1 g/100 mL or more relative to the entire tungsten raw material in step (i).

### (Condition (b))

A peak (peak E) observed at 2θ=27.1±0.2° in powder X-ray measurement performed with a temperature rise of the catalyst containing heteropoly acid including phosphorus, molybdenum and tungsten originates from molybdenum oxide generated by heteropoly acid decomposition or the like. In the catalyst for methacrylic acid production according to the present embodiment, the lowest temperature T1 is 440° C. or more, at which the peak D appears in powder X-ray diffraction measurement performed at an interval of 10° C. from 400° C. with a temperature rise at 10° C./min. The catalyst having a T1 of 440° C. or more means the catalyst in which heteropoly acid forms a thermodynamically metastable crystal structure. It is considered that the heteropoly acid here is high in heat resistance and has an active point suitable for oxidation reaction of methacrolein. The lower limit of the T1 is preferably 450° C. or more, more preferably 460° C. or more, further preferably 470° C. or more. The upper limit of the T1 is preferably 500° C. or less, more preferably 490° C. or less, further preferably 480° C. or less.

Examples of the method for providing a catalyst having T1 within a prescribed range include a method including production of the catalyst by a method including steps (i) to (iii) described below and adjustment of the amounts of the tungsten raw material and the raw material of the element G used in step (i).

For example, an X-ray diffraction apparatus X'PertProMPD manufactured by PANalytical and a high-temperature strip heater chamber manufactured by Anton Paar GmbH can be used for powder X-ray measurement of the catalyst. In the measurement, CuKα ray (λ=0.154 nm) is used, the output power is set to 45 kV and 40 mA, and the measurement range is set to 5 to 60°. The goniometer radius is 240 mm, and the divergent slit here used is an automatic continuously variable slit or a fixed slit. When the automatic continuously variable slit is used, the slit size is 0 to 8.0 mm, and when the fixed slit is used, the slit size is 1.75 mm and the slit open angle is 1.00°. The resulting X-ray diffraction pattern is subjected to separation of the Kα2 line and thus peak search, and the IA, IB, IC and ID are determined. Peak search conditions are as follows: minimum degree of significance: 1 degree, minimum peak tip: 0.2 degrees, maximum peak tip: 1 degree, peak base width: 2 degrees, and method: minimum value in secondary differentiation. The peak search can be performed with, for example, High Score Plus manufactured by Panalytical. The lowest temperature at which the peak E appears is the lowest temperature at which a peak is detected at 2θ=27.1±0.2° when the X-ray diffraction pattern obtained by performing powder X-ray diffraction measurement of the catalyst at an interval of 10° C. from 400° C. with a temperature rise at 10° C./min is subjected to peak search by the above method.

### Composition of Catalyst

The catalyst for methacrylic acid production according to the present embodiment preferably has a composition represented by the following formula (I) from the viewpoint of the yield of methacrylic acid. The catalyst may include a small amount of any element not described in the following formula (I).

PₐMo_{b}W_{c}V_{d}CuₑA_{f}E_{g}GₕOᵢ (I)

In the formula (I), P, Mo, W, V, Cu and O respectively represent phosphorus, molybdenum, tungsten, vanadium, copper, and oxygen. A represents at least one element selected from the group consisting of antimony, bismuth, arsenicum, germanium, tellurium, selenium, and silicon. E represents at least one element selected from the group consisting of iron, zinc, chromium, tantalum, cobalt, nickel, manganese, titanium and niobium. G represents at least one element selected from the group consisting of potassium, rubidium and cesium. a to i each represent the molar ratio of each component, b+c=12, a=0.5 to 3, c=0.22 to 5, d=0.01 to 3, e=0.01 to 2, f=0 to 3, g=0 to 3, and h=0.01 to 3 are satisfied, and i is the molar ratio of oxygen necessary for satisfying the valence of such each component.

In the formula (I), c as the molar ratio of W (tungsten) satisfies c=0.22 to 5. Thus, a catalyst in which the conditions (a1) and (b) are satisfied can be easily obtained. The lower limit of c is preferably 0.23 or more, more preferably 0.3 or more. The upper limit of c is preferably 3 or less, more preferably 2 or less, further preferably 1 or less, particularly preferably 0.8 or less.

In the formula (I), from the viewpoint of an enhancement in yield of methacrylic acid, the lower limit of a is preferably 0.6 or more, more preferably 0.7 or more. The upper limit of a is preferably 2.5 or less, more preferably 2 or less. The lower limit of d is preferably 0.1 or more, more preferably 0.15 or more, further preferably 0.2 or more. The upper limit of d is preferably 2.5 or less, more preferably 2 or less, further preferably 1.5 or less. The lower limit of e is preferably 0.03 or more, more preferably 0.05 or more. The upper limit of e is preferably 2.5 or less, more preferably 2 or less. The lower limit of f is preferably 0.01 or more, more preferably 0.1 or more. The upper limit of f is preferably 2.5 or less, more preferably 2 or less. The upper limit of g is preferably 2 or less, more preferably 1.5 or less, further preferably 1 or less. The lower limit of h is preferably 0.1 or more, more preferably 0.3 or more. The upper limit of h is preferably 2.8 or less, more preferably 2.5 or less.

The molar ratio of each component is a value determined by analyzing such each component of the catalyst dissolved in ammonia water, with ICP emission spectroscopy.

### Method for Producing Catalyst for Methacrylic Acid Production

The catalyst for methacrylic acid production according to the present embodiment can be produced according to a known catalyst production method as long as phosphorus, molybdenum and tungsten are included and the conditions (a1) and (b) are satisfied, and the catalyst is preferably produced by a method including the following steps (i) to (iii).
(i) A step of mixing a phosphorus raw material, a molybdenum raw material and a tungsten raw material with a solvent, to prepare a solution or slurry (liquid A) having a pH of 0.1 to 4.
(ii) A step of drying the liquid A, to obtain a dried product.
(iii) A step of firing the dried product, to obtain a fired product.

The method for producing the catalyst for methacrylic acid production according to the present embodiment may further include a molding step described below.

Hereinafter, each step is described in detail.

<Step (i)>

In step (i), a phosphorus raw material, a molybdenum raw material and a tungsten raw material are mixed with a solvent, to prepare a solution or slurry (liquid A) having a pH of 0.1 to 4. The liquid A may be mixed with a raw material of any element other than phosphorus, molybdenum and tungsten in the formula (I), and is preferably mixed with a raw material of the element G.

The liquid A can be prepared by dissolving or suspending raw materials of catalyst components including the phosphorus raw material, the molybdenum raw material and the tungsten raw material, in a solvent.

### Raw Materials of Catalyst Components

The raw materials of the catalyst component are not particularly limited, and nitrate, carbonate, acetate, an ammonium salt, oxide, halide, oxyacid, an oxyacid salt, and/or the like of each constituent element of the catalyst can be used singly or in combination of two or more kinds thereof.

Examples of the phosphorus raw material include phosphoric acid, phosphorus pentoxide, and ammonium phosphate. Examples of the molybdenum raw material include molybdenum oxides such as molybdenum trioxide, ammonium molybdates such as ammonium paramolybdate and ammonium dimolybdate, and molybdenum chloride.

Examples of the tungsten raw material include tungsten oxide, sodium tungstate, phosphotungstic acid, and ammonium metatungstate, and a tungsten raw material having a solubility at 20° C. of 4.1 g/100 mL or more preferably occupies 50% by mass or more of the entire tungsten raw material. Thus, a catalyst in which conditions (a1) and (b) are satisfied can be easily obtained. Examples of such a tungsten raw material having a solubility at 20° C. of 4.1 g/100 mL or more include phosphotungstic acid and ammonium metatungstate. Such a tungsten raw material having a solubility at 20° C. of 4.1 g/100 mL or more occupies more preferably 70% by mass or more, further preferably 90% by mass or more of the tungsten raw material.

In a case where a catalyst further including vanadium and copper is produced, examples of a vanadium raw material include ammonium metavanadate, vanadium pentoxide, vanadium chloride, and vanadyl oxalate. Examples of a copper raw material include copper sulfate, copper nitrate, copper oxide, copper carbonate, copper acetate, and copper chloride.

The concentration of the raw materials of the catalyst components in the liquid A is not particularly limited, and is preferably within a range of 5 to 90% by mass.

### Solvent

Examples of the solvent include water, ethyl alcohol, and acetone. These may be used singly or in combination of two or more kinds thereof. In particular, water is preferably used from an industrial viewpoint.

### Preparation of Liquid A

The liquid A is preferably prepared with a preparation container by adding the raw materials of the catalyst components to the solvent and stirring them with heating. Thus, heteropoly acid suitable for production of methacrylic acid is sufficiently produced.

The preparation can be usually performed at a heating temperature in the range of 30 to 150° C., and is preferably performed at a heating temperature in the range of 60 to 150° C. When the heating temperature is 60° C. or more, the rate of generation of the heteropoly acid can be sufficiently increased, and when the heating temperature is 150° C. or less, evaporation of the solvent can be suppressed. The lower limit of the heating temperature is more preferably 80° C. or more, further preferably 90° C. or more. The upper limit of the heating temperature is more preferably 130° C. or less, further preferably 110° C. or less. In addition, concentration and/or reflux may be made during heating, depending on the vapor pressure of the solvent used, or heating treatment may be made under a pressurized condition by operation in a closed container.

The rate of temperature rise is not particularly limited, and is preferably 0.8 to 15° C./min. When the rate of temperature is 0.8° C./min or more, the time necessary for step (i) can be shortened. When the rate of temperature rise can be 15° C./min or less, temperature rise can be made with usual temperature rise equipment.

The stirring is preferably performed at a stirring power of 0.01 kW/m³ or more, more preferably 0.05 kW/m³ or more. When the stirring power is 0.01 kW/m³ or more, local irregularities of the temperature of the liquid A, the component, and the temperature are decreased and a structure suitable in a catalyst for methacrylic acid production is stably formed. The stirring is usually preferably performed at a stirring power of 3.5 kW/m³ or less from the viewpoint of production cost of the catalyst.

### Physical Properties of Liquid A

Preferably, the pH of the liquid A is 0.1 to 4, and the lower limit thereof is 0.5 or more and the upper limit thereof is 3 or less. Thus, production reaction of heteropoly acid suitable for methacrylic acid production is stabilized. Examples of the method for allowing the pH of the liquid A to be 0.1 to 4 include a method in which molybdenum trioxide is used as the molybdenum raw material or a method in which a raw material compound is appropriately selected to adjust the content of nitric acid ions or oxalic acid ions.

<Step (ii)>

In step (ii), the liquid A obtained in step (i) is spray-dried, to obtain a dried product.

Examples of the drying method include known methods such as a drum drying method, a flash drying method, an evaporation drying method, and a spray drying method. In particular, a spray drying method is preferably used because a particulate dried product is obtained and the shape of the dried product is a neat spherical shape.

The drying temperature differs depending on the drying method, and can be usually 100 to 500° C., and the lower limit is preferably 140° C. or more and the upper limit is preferably 400° C. or less. The drying is preferably performed so that the content rate of moisture in the resulting dried product is 4.5% by mass or less, more preferably 0.1 to 4.5% by mass. These conditions are not particularly limited, and can be appropriately selected depending on the desired shape and size of the dried product.

The dried product obtained in step (ii) may be, if necessary, subjected to molding described below.

### <Molding Step>

In the molding step, the dried product obtained in step (ii) is, if necessary, molded. Here, the molding may be performed after step (iii) described below.

The molding method is not particularly limited, any known dry or wet molding method can be applied, and examples include tablet molding, press molding, extrusion molding, and granulation molding. The shape of a molded product is not particularly limited, and examples include a columnar shape, a ring shape, and a spherical shape. When the molding is made, only the dried product is preferably molded without addition of any carrier or the like to the dried product, and, for example, a known additive such as graphite or talc may be, if necessary, added. In a case where a carrier is used, the carrier is not particularly limited, and examples preferably include silica.

<Step (iii)>

In the firing step, the dried product obtained in step (iii), and the dried product after molding, obtained in the molding step, are fired, to obtain a fired product.

The firing can be performed under flow of at least one of an oxygen-containing gas such as air, and an inert gas, and the firing is preferably performed under flow of an oxygen-containing gas such as air. The inert gas here refers to a gas causing no reduction in catalyst activity, and examples include nitrogen, carbon dioxide gas, helium, and argon. These may be used singly, or as a mixture of two or more kinds thereof.

The shape of a firing container is not particularly limited, and, for example, a box-type or tubular container can be used. The firing can also be performed with division into a plurality of containers and filling. In particular, a tubular container having a cross-sectional area of 1 to 100 cm² is preferably used.

The firing temperature (the highest temperature during the firing) is preferably 200 to 700° C., and the lower limit is more preferably 320° C. or more, and the upper limit is more preferably 450° C. or less.

The fired product obtained above can be used as the catalyst for methacrylic acid production. The fired product may be molded as described with respect to the molding step. In the present embodiment, the fired product, and the fired product after the molding are collectively referred to as “catalyst”.

### Method for Producing Methacrylic Acid

A method for producing methacrylic acid according to the present embodiment includes oxidizing methacrolein with the catalyst for methacrylic acid production according to the present embodiment. The method for producing methacrylic acid according to the present embodiment includes oxidizing methacrolein with a catalyst for methacrylic acid production, produced by the production method according to the present embodiment. According to such a method, methacrylic acid can be produced at a high yield.

The method for producing methacrylic acid according to the present embodiment can be carried out by contacting the catalyst for methacrylic acid production according to the present embodiment and a raw material gas containing methacrolein. A fixed bed-type reactor can be used in this reaction. This reaction can be performed by filling the reactor with the catalyst, and feeding the raw material gas to the reactor. A catalyst layer may be a single layer, or a plurality of such catalysts different in activity may be divided respectively to a plurality of layers and subjected to filling. The catalyst may be diluted with an inert carrier and subjected to filling in order to control activity.

The concentration of methacrolein in the raw material gas is preferably 1 to 20% by volume, the lower limit thereof is more preferably 3% by volume or more, and the upper limit thereof is more preferably 10% by volume or less. Methacrolein as a raw material may include a small amount of impurities having no substantial influence on the present reaction of lower saturated aldehyde or the like.

The oxygen source of the raw material gas is not particularly limited, and air is preferably used from an industrial viewpoint. A gas in which pure oxygen is mixed with air or the like can also be, if necessary, used. The proportion of oxygen in the raw material gas is not particularly limited, the proportion based on 1 mol of methacrolein is preferably 0.4 to 4 mol, the lower limit thereof is more preferably 0.5 mol or more, and the upper limit thereof is more preferably 3 mol or less.

The raw material gas may be one diluted with an inert gas such as nitrogen or carbon dioxide from the viewpoint of economic performance. Water vapor may also be further added to the raw material gas. Methacrylic acid can be obtained at a high yield by reaction performed in the presence of water vapor. The concentration of water vapor in the raw material gas is preferably 0.1 to 50% by volume, the lower limit is more preferably 1% by volume or more, and the upper limit is more preferably 40% by volume.

The contact time between the raw material gas and the catalyst for methacrylic acid production is preferably 1.5 to 15 seconds, the lower limit is more preferably 2 seconds or more, and the upper limit is more preferably 10 seconds or less. The reaction pressure is preferably 0.1 to 1 MPa (G). Herein, (G) means the gauge pressure. The reaction temperature is preferably 200 to 450° C., and the lower limit is more preferably 250° C. or more and the upper limit is more preferably 400° C. or less.

In the production of methacrylic acid, the location of the catalyst in the reactor, the proportion of the catalyst in the reactor, and the like are not particularly limited, and any form commonly used can be applied.

### Method for Producing Methacrylic Acid Ester

A method for producing methacrylic acid ester according to the present embodiment includes esterifying methacrylic acid produced by the production method according to the present embodiment. In other words, the method for producing methacrylic acid ester according to the present embodiment includes a step of producing methacrylic acid by the production method according to the present embodiment and a step of esterifying the methacrylic acid.

The alcohol to be allowed to react with the methacrylic acid is not particularly limited, and examples include methanol, ethanol, n-propanol, isopropanol, n-butanol, and isobutanol. Examples of the methacrylic acid ester obtained include methyl methacrylate, ethyl methacrylate, propyl methacrylate, isopropyl methacrylate, butyl methacrylate, and isobutyl methacrylate. Such esterification reaction can be performed in the presence of an acidic catalyst such as a sulfonic acid-type cation-exchange resin. The temperature in the esterification reaction is preferably 50 to 200° C.

The pressure in the esterification reaction, the location of the catalyst in the reactor, the proportion of the catalyst in the reactor, and the like are not particularly limited, and any form commonly used can be applied.

### EXAMPLES

Hereinafter, Production Examples of the catalyst according to the present embodiment, and Reaction Examples with the catalyst are described together with Comparative Examples. In the following Examples and Comparative Examples, “part(s)” means part(s) by mass.

### Composition Ratio of Catalyst

The molar ratio of each component was determined by analyzing such each component of the catalyst dissolved in ammonia water, with ICP emission spectroscopy.

### Powder X-Ray Diffraction Measurement of Catalyst

An X-ray diffraction apparatus X'PertProMPD manufactured by PANalytical and a high-temperature strip heater chamber manufactured by Anton Paar GmbH were used in powder X-ray diffraction measurement of the catalyst. In the measurement, CuKα ray (λ=0.154 nm) was used, the output power was set to 45 kV and 40 mA, and the measurement range was 5 to 60°. The goniometer radius was 240 mm, and the divergent slit here used was an automatic continuously variable slit or a fixed slit. When the automatic continuously variable slit was used, the slit size was 0 to 8.0 mm, and when the fixed slit was used, the slit size was 1.75 mm, and the slit open angle was 1.00°. The resulting X-ray diffraction pattern was subjected to separation of the Kα2 line and thus peak search by use of High Score Plus manufactured by Panalytical, and the IA, IB, IC and ID were determined. Peak search conditions were as follows: minimum degree of significance: 1 degree, minimum peak tip: 0.2 degrees, maximum peak tip: 1 degree, peak base width: 2 degrees, and method: minimum value in secondary differentiation. The X-ray diffraction pattern obtained by performing powder X-ray diffraction measurement of the catalyst at an interval of 10° C. from 400° C. with a temperature rise at 10° C./min was subjected to peak search by the above method, and the lowest temperature at which a peak was detected at 2θ=27.1±0.2° was defined as T1.

### Pyrolysis Temperature of Catalyst

The pyrolysis temperature as an index of heat resistance of the catalyst was measured with a TG/DTA measurement apparatus, as follows. Fifty mg of alumina was used as reference, and 50 mg of a powdery catalyst was heated from room temperature to 550° C. at 10° C./min in an air atmosphere. The exothermic onset temperature at which exotherm corresponding to 1.8 μV or more at a temperature rise of 15° C. or more was observed in a temperature region of 380° C. or more in the resulting DTA curve was defined as the pyrolysis temperature. It is meant that, as the pyrolysis temperature of the catalyst is higher, heat resistance is higher.

### Analysis of Raw Material Gas and Product

The raw material gas and the product were analyzed with the following gas chromatography.

GC-2014 manufactured by Shimadzu Corporation (column: DB-FFAP manufactured by J&W, 30 m×0.32 mm, film thickness 1.0 μm)

GC-8A manufactured by Shimadzu Corporation (column: Molecular Sieve, 2.0 M×3.0 mm ID)

GC-8A manufactured by Shimadzu Corporation (column: Porapak Q, 2.0 M×3.0 mm ID)

The yield of methacrylic acid produced was calculated by the following formula.

Yield of methacrylic acid (%)=(N2/N1)×100

Here, N1 represents the number of moles of methacrolein fed, and N2 represents the number of moles of methacrylic acid produced.

### Tungsten Raw Material

Tungsten raw materials used in Examples and Comparative Examples are as follows.

Ammonium metatungstate (manufactured by NIPPON INORGANIC COLOUR & CHEMICAL CO., LTD.) Solubility in water at 20° C.: 10 g/100 mL

Phosphotungstic acid (manufactured by NIPPON INORGANIC COLOUR & CHEMICAL CO., LTD.) Solubility in water at 20° C.: 40 g/100 mL or more

### Example 1

A diluted product obtained by diluting 294.0 parts of molybdenum trioxide, 10.2 parts of ammonium metavanadate, 10.6 parts of ammonium metatungstate, and 30.0 parts of an aqueous 85% by mass phosphoric acid solution with 36 parts of pure water, and a dissolved product obtained by dissolving 6.3 parts of copper (II) nitrate trihydrate in 9.0 parts of pure water were mixed with 1200 parts of pure water at room temperature. The resulting slurry was heated to 95° C. at 2° C./min, and stirred for 2 hours. Next, a dissolved product obtained by dissolving 40.4 parts of cesium bicarbonate in 60 parts of pure water at room temperature was mixed, and stirred at 95° C. for 15 minutes. Next, a dissolved product obtained by dissolving 27.5 parts of ammonium carbonate in 78 parts of pure water was mixed, and stirred at 95° C. for 15 minutes, and thus a liquid A was obtained. The pH of the liquid A is shown in Table 1.

The liquid A obtained was heated, and evaporated to dryness, and thus a dried product was obtained.

The dried product obtained was pressure molded, ground, and classified with a sieve so that the particle size was within a range of 710 μm to 2.36 mm. The resulting ground object was fired at 380° C. for 5 hours under flow of air, and the resulting fired product was adopted as a catalyst. The composition excluding oxygen, of the catalyst, satisfied P_{1.5}Mo_{11.8}W_{0.2}V_{0.5}Cu_{0.15}Cs_{1.2}. The catalyst was subjected to powder X-ray diffraction measurement and pyrolysis temperature measurement. The results are shown in Table 1.

A fixed bed flow type reactor was filled with 8 g of the resulting catalyst diluted with 10 g of silicon carbide. Next, a raw material gas including 5% by volume of methacrolein, 10% by volume of oxygen, 30% by volume of water vapor and 55% by volume of nitrogen was allowed to flow for a contact time of 178 hr·g/mol, to perform oxidation reaction of methacrolein at a reaction temperature of 300° C. The reaction performance is shown in Table 1.

### Example 2

A liquid A was obtained by the same method as in Example 1 except that 292.1 parts of molybdenum trioxide and 13.6 parts of ammonium metatungstate were used. The pH of the liquid A is shown in Table 1.

The liquid A obtained was heated, and evaporated to dryness, and thus a dried product was obtained.

The dried product obtained was used, and a catalyst was obtained by the same method as in Example 1. The composition excluding oxygen, of the catalyst, satisfied P_{1.5}Mo_{11.7}W_{0.3}Cu0.15Cs 1.2. The catalyst was subjected to powder X-ray diffraction measurement and pyrolysis temperature measurement. The results are shown in Table 1.

The catalyst obtained was used, and oxidation reaction of methacrolein was performed by the same method as in Example 1. The reaction performance is shown in Table 1.

### Example 3

A liquid A was obtained by the same method as in Example 1 except that 287.1 parts of molybdenum trioxide and 22.6 parts of ammonium metatungstate were used. The pH of the liquid A is shown in Table 1.

The liquid A obtained was heated, and evaporated to dryness, and thus a dried product was obtained.

The dried product obtained was used, and a catalyst was obtained by the same method as in Example 1. The composition excluding oxygen, of the catalyst, satisfied P_{1.5}Mo_{11.5}W_{0.5}V_{0.5}Cu_{0.15}Cs_{1.2}. The catalyst was subjected to powder X-ray diffraction measurement and pyrolysis temperature measurement. The results are shown in Table 1.

The catalyst obtained was used, and oxidation reaction of methacrolein was performed by the same method as in Example 1. The reaction performance is shown in Table 1.

### Example 4

A liquid A was obtained by the same method as in Example 1 except that 285.0 parts of molybdenum trioxide and 29.0 parts of an aqueous 85% by mass phosphoric acid solution were used, and 29.7 parts of phosphotungstic acid was used instead of 10.6 parts of ammonium metatungstate. The pH of the liquid A is shown in Table 1.

The liquid A obtained was heated, and evaporated to dryness, and thus a dried product was obtained.

The dried product obtained was used, and a catalyst was obtained by the same method as in Example 1. The composition excluding oxygen, of the catalyst, satisfied P_{1.5}Mo_{11.4}W_{0.6}V_{0.5}Cu_{0.15}Cs_{1.2}. The catalyst was subjected to powder X-ray diffraction measurement and pyrolysis temperature measurement. The results are shown in Table 1.

The catalyst obtained was used, and oxidation reaction of methacrolein was performed by the same method as in Example 1. The reaction performance is shown in Table 1.

### Example 5

A liquid A was obtained by the same method as in Example 1 except that 277.2 parts of molybdenum trioxide and 40.8 parts of ammonium metatungstate were used. The pH of the liquid A is shown in Table 1.

The liquid A obtained was heated, and evaporated to dryness, and thus a dried product was obtained.

The dried product obtained was used, and a catalyst was obtained by the same method as in Example 1. The composition excluding oxygen, of the catalyst, satisfied P_{1.5}Mo_{11.1}W_{0.9}V_{0.5}Cu_{0.15}Cs_{1.2}. The catalyst was subjected to powder X-ray diffraction measurement and pyrolysis temperature measurement. The results are shown in Table 1.

The catalyst obtained was used, and oxidation reaction of methacrolein was performed by the same method as in Example 1. The reaction performance is shown in Table 1.

### Comparative Example 1

A liquid A was obtained by the same method as in Example 1 except that 270.0 parts of molybdenum trioxide and 53.2 parts of ammonium metatungstate were used. The pH of the liquid A is shown in Table 1.

The liquid A obtained was heated, and evaporated to dryness, and thus a dried product was obtained.

The dried product obtained was used, and a catalyst was obtained by the same method as in Example 1. The composition excluding the oxygen, of catalyst, satisfied P_{1.5}Mo_{10.8}W_{1.2}V_{0.5}Cu_{0.15}Cs_{1.2}. The catalyst was subjected to powder X-ray diffraction measurement and pyrolysis temperature measurement. The results are shown in Table 1.

The catalyst obtained was used, and oxidation reaction of methacrolein was performed by the same method as in Example 1. The reaction performance is shown in Table 1.

### Comparative Example 2

A liquid A was obtained by the same method as in Example 1 except that 270.0 parts of molybdenum trioxide and 27.4 parts of an aqueous 85% by mass phosphoric acid solution were used, and 59.4 parts of phosphotungstic acid was used instead of 10.6 parts of ammonium metatungstate. The pH of the liquid A is shown in Table 1.

The liquid A obtained was heated, and evaporated to dryness, and thus a dried product was obtained.

The dried product obtained was used, and a catalyst was obtained by the same method as in Example 1. The composition excluding oxygen, of the catalyst, satisfied P_{1.5}Mo_{10.8}W_{1.2}V_{0.5}Cu_{0.15}Cs_{1.2}. The catalyst was subjected to powder X-ray diffraction measurement and pyrolysis temperature measurement. The results are shown in Table 1.

The catalyst obtained was used, and oxidation reaction of methacrolein was performed by the same method as in Example 1. The reaction performance is shown in Table 1.

### Comparative Example 3

300.0 parts of phosphotungstic acid was pressure molded, ground, and classified with a sieve so that the particle size was within a range of 710 μm to 2.36 mm. The resulting ground object was fired at 380° C. for 5 hours under flow of air, and the resulting fired product was adopted as a catalyst. The composition excluding oxygen, of the catalyst, satisfied P₁W₁₂.

The catalyst was subjected to powder X-ray diffraction measurement and pyrolysis temperature measurement. The results are shown in Table 1.

The catalyst obtained was used, and oxidation reaction of methacrolein was performed by the same method as in Example 1. The reaction performance is shown in Table 1.

### Comparative Example 4

A powder parts of obtained by firing 300.0 phosphomolybdic acid (manufactured by NIPPON INORGANIC COLOUR & CHEMICAL CO., LTD.) at 380° C. for 5 hours was ground, and classified with a sieve so that the particle size was within a range of 710 μm to 2.36 mm. The resulting ground object was fired at 380° C. for 5 hours under flow of air, and thus a catalyst was produced. The composition excluding oxygen, of the catalyst, satisfied P₁MO₁₂. The catalyst was subjected to powder X-ray diffraction measurement and pyrolysis temperature measurement. The results are shown in Table 1.

The catalyst obtained was used, and oxidation reaction of methacrolein was performed by the same method as in Example 1. The reaction performance is shown in Table 1.

TABLE 1

| | | | | | | | | Condition | Condition | Condition | Condition | Pyrolysis | Yield of |
|---|---|---|---|---|---|---|---|---|---|---|---|---|---|
| | Compositional ratio of catalyst | | | | | | pH of | (a1) | (a2) | (a3) | (b) | temperature | methacrylic |
| | a | b | c | d | e | h | liquid A | IB/IA | IC/IA | ID/IA | T1 [° C.] | [° C.] | acid [%] |
| Example1 | 1.5 | 11.8 | 0.2 | 0.5 | 0.15 | 1.2 | 1.9 | 0 | 0.0052 | 0 | 480 | 442 | 49.6 |
| Example2 | 1.5 | 11.7 | 0.3 | 0.5 | 0.15 | 1.2 | 2.7 | 0 | 0 | 0 | 460 | 447 | 50.6 |
| Example3 | 1.5 | 11.5 | 0.5 | 0.5 | 0.15 | 1.2 | 2.8 | 0 | 0 | 0 | 470 | 446 | 45.3 |
| Example4 | 1.5 | 11.4 | 0.6 | 0.5 | 0.15 | 1.2 | 2.3 | 0 | 0.0040 | 0 | 470 | 439 | 50.5 |
| Example5 | 1.5 | 11.1 | 0.9 | 0.5 | 0.15 | 1.2 | 3.0 | 0 | 0 | 0 | 480 | 451 | 34.8 |
| Comparative | 1.5 | 10.8 | 1.2 | 0.5 | 0.15 | 1.2 | 3.2 | 0.0182 | 0.0068 | 0.0031 | 480 | 453 | 7.6 |
| Example1 | | | | | | | | | | | | | |
| Comparative | 1.5 | 10.8 | 1.2 | 0.5 | 0.15 | 1.2 | 1.8 | 0 | 0.0057 | 0.0149 | 480 | 439 | 33.2 |
| Example2 | | | | | | | | | | | | | |
| Comparative | 1 | 0 | 12 | 0 | 0 | 0 | — | 0.5697 | 0.3702 | 0.0679 | 550 | 600 | 1.3 |
| Example3 | | | | | | | | | | | | | |
| Comparative | 1 | 12 | 0 | 00 | 0 | 0 | — | 0 | 0.1023 | 0.1211 | 420 | 416 | 13.9 |
| Example4 | | | | | | | | | | | | | |

As shown in Table 1, each of Examples 1 to 5, in which the catalyst satisfying the conditions (a1) and (b) was used, provided the catalyst having high heat resistance and allowing for production of methacrylic acid at a high yield because the catalyst in which heteropoly acid formed a thermodynamically metastable crystal structure.

Here, methacrylic acid obtained in the present Examples can be esterified, thereby obtaining methacrylic acid ester.

### INDUSTRIAL APPLICABILITY

According to the present invention, there can be provided a catalyst which has high heat resistance and which can allow for production of methacrylic acid at a high yield.

1. A catalyst for use in the production of methacrylic acid by oxidation of methacrolein, wherein
the catalyst contains heteropoly acid including phosphorus, molybdenum and tungsten,
(a1) IB/IA is 0.015 or less when the intensity of a diffraction peak (peak A) at 2θ=26.1±0.2° is defined as IA and the intensity of a diffraction peak (peak B) at 2θ=23.0±0.2° is defined as IB in powder X-ray diffraction measurement at 25° C., and
(b) the lowest temperature T1 at which a diffraction peak (peak E) at 2θ=27.1±0.2° appears in powder X-ray diffraction measurement performed at an interval of 10° C. from 400° C. with a temperature rise at 10° C./min is 440° C. or more.

2. The catalyst for methacrylic acid production according to claim 1, wherein the IB/IA is 0.01 or less.

3. The catalyst for methacrylic acid production according to claim 1, wherein (a2) IC/IA is 0.0055 or less when the intensity of a diffraction peak (peak C) at 2θ=34.1±0.2° is defined as IC in powder X-ray diffraction measurement at 25° C.

4. The catalyst for methacrylic acid production according to claim 3, wherein the IC/IA is 0.0005 to 0.0055.

5. The catalyst for methacrylic acid production according to claim 3, wherein the IC/IA is 0.001 to 0.0053.

6. The catalyst for methacrylic acid production according to claim 1, wherein (a3) ID/IA is 0.003 or less when the intensity of a diffraction peak (peak D) at 2θ=48.3±0.2° is defined as ID in powder X-ray diffraction measurement at 25° C.

7. The catalyst for methacrylic acid production according to claim 1, wherein the T1 is 440 to 500° C.

8. The catalyst for methacrylic acid production according to claim 1, having a composition represented by the following formula (I):
PₐMo_{b}W_{c}V_{d}CuₑA_{f}E_{g}GₕOᵢ (I)
wherein P, Mo, W, V, Cu and O respectively represent phosphorus, molybdenum, tungsten, vanadium, copper and oxygen, A represents at least one element selected from the group consisting of antimony, bismuth, arsenicum, germanium, tellurium, selenium and silicon, E represents at least one element selected from the group consisting of iron, zinc, chromium, tantalum, cobalt, nickel, manganese, titanium and niobium, G represents at least one element selected from the group consisting of potassium, rubidium and cesium, a to i each represent the molar ratio of each component, b+c=12, a=0.5 to 3, c=0.22 to 5, d=0.01 to 3, e=0.01 to 2, f=0 to 3, g=0 to 3, and h=0.01 to 3 are satisfied, and i is the molar ratio of oxygen necessary for satisfying the valence of such each component.

9. The catalyst for methacrylic acid production according to claim 8, wherein c=0.22 to 3 is satisfied in the formula (I).

10. A method for producing the catalyst for methacrylic acid production according to claim 1, the method comprising:
(i) a step of mixing a phosphorus raw material, a molybdenum raw material and a tungsten raw material with a solvent, to prepare a solution or slurry (liquid A) having a pH of 0.1 to 4;
(ii) a step of drying the liquid A, to obtain a dried product; and
(iii) a step of firing the dried product, to obtain a fired product.

11. The method for producing the catalyst for methacrylic acid production according to claim 10, wherein a tungsten raw material having a solubility at 20° C. of 4.1 g/100 mL or more occupies 50% by mass or more of the entire tungsten raw material in step (i).

12. The method for producing the catalyst for methacrylic acid production according to claim 10, wherein the pH of the liquid A is 0.1 to 3 in step (i).

13. A method for producing methacrylic acid, including a step of oxidizing methacrolein with the catalyst for methacrylic acid production according to claim 1, to produce methacrylic acid.

14. A method for producing methacrylic acid, comprising a step of oxidizing methacrolein with a catalyst for methacrylic acid production, produced by the method according to claim 10, to produce methacrylic acid.

15. A method for producing methacrylic acid ester, comprising a step of esterifying methacrylic acid produced by the method according to claim 13.
